(19) United States
(12) Patent Application Publication
(10) Pub. No.: US 2006/0222585 A1
(43) Pub. Date: Oct. 5, 2006

(54) Production of organic acid and ammonium nitrate

(75) Inventors: Dan Verser, Golden, CO (US); Tim Eggeman, Lakewood, CO (US)

Correspondence Address: SHERIDAN ROSS PC, 1560 BROADWAY, SUITE 1200, DENVER, CO, 80202, US

(73) Assignee: ZEACHEM, INC., Golden, CO (US)

(21) Appl. No.: 10/541,801

(22) PCT Filed: Jan. 9, 2004
(86) PCT No.: PCT/US04/00402

§ 371 Date: Feb. 3, 2006

Related U.S. Application Data
(60) Provisional application No. 60/439,148, filed on Jan. 10, 2003.

(51) Int. Cl. C12P 7/40; C01C 1/18; C07C 51/42

(52) U.S. Cl. 423/396; 435/136; 562/494; 562/590

(57) ABSTRACT

A process for the recovery of organic acids from dilute solutions such as those produced by fermentation, when the organic acids are present as dilute salt solutions, is provided. The organic acid production process is integrated with a nitrogen fertilizer production process by utilizing wasted chemical energy from the fertilizer process for acidification of the organic acid solution.

### FIELD OF THE INVENTION

This invention relates to a process for the recovery of organic acids from dilute solutions such as those produced by fermentation, when the organic acids are present as dilute salt solutions.

### BACKGROUND OF THE INVENTION

Organic acids are valuable chemicals used in industrial and food ingredient applications and as intermediates in the synthesis of other chemical products. They may be produced by fermentation by a wide range of organisms.

Typically for the production of organic acids by fermentation the broth produced in the fermentation actually contains the organic acid in the form of a salt rather than in its protonated acid form since the fermentation is conducted at a near neutral pH which is required by the organisms, and the pKa of important organic acids are well below neutral.

The salt form is typically highly water soluble, has a high boiling point and the carbonyl group is unreactive. These properties make recovery of organic acid salts difficult since distillation, extraction, reactive separation and other common industrial separation methods for large scale production are either technically or economically infeasible. One way to ease the recovery of organic acid salts is to add a mineral acid to lower the pH of the broth, thereby converting the organic acid into its protonated form. In its protonated form the organic acid can be more easily recovered by known means such as distillation, extraction or reactive separation processes.

Direct acidification with a mineral acid is usually regarded as a troublesome option for recovery of organic acid salts because a salt byproduct is inevitably formed. This byproduct is often of very low value. For example, gypsum is historically the salt co-produced in lactic acid production.

Calcium lactate+sulfuric acid+calcium sulfate+lactic acid (Ref. Holten, C. H., Lactic Acid: Properties and Chemistry of Lactic Acid and Derivatives, Verlag Chemie, 1971.)

Markets either have to be found or an environmentally responsible disposal method has to be identified. Because of these limitations, much research has gone into alternative methods to recover organic acids. However, direct acidification remains a simple and well-understood process if its limitations can be overcome.

Fertilizer use, especially of nitrogen-based fertilizers, is a key aspect of modern high yield agricultural practice. Nitrogen is incorporated into plant proteins and nucleic acids, and thus is considered a primary nutrient. Plants are incapable of directly fixing atmospheric nitrogen. For non-legumes, nitrogen has to be supplied in a fixed form through soil organic matter, ammonium ions, nitrate ions, etc. Legumes, such as soybeans, have a symbiotic relationship with diazotrophic soil bacteria that are capable of fixing atmospheric nitrogen, thus supplying some of the nitrogen needs for the plant. However, the high yields of today's legume crops are only obtained with additional nitrogen supplementation from fertilizers.

FIG. 1 is a simplified block flow diagram for a typical nitrogen fertilizer complex using today's technology. Natural gas is converted into syngas, which is further processed into hydrogen and carbon dioxide. The hydrogen is used to produce ammonia. Ammonia is used as a feedstock for nitric acid production. The resulting nitric acid is neutralized with additional ammonia to produce ammonium nitrate. Ammonia is also reacted with carbon dioxide to produce urea. All three nitrogen products are used, either directly or in combination through formulation, as fertilizers.

The present invention uses ammonium nitrate as a target for the salt coproduct in the direct acidification process for organic acid production. Ammonium nitrate is an appealing target because U.S. demand for ammonium nitrate is forecasted at 3,350 thousand metric tons (nitrogen basis) in 2004, mostly for nitrogen fertilizer.

### SUMMARY OF THE INVENTION

The present invention is directed to a process for the production and recovery of organic acids from dilute salt solutions, such as those produced by fermentation, which is integrated with the production of nitrogen fertilizers in order to capture wasted chemical energy from the fertilizer process.

The present invention is directed to a method for production of an organic acid and ammonium nitrate. The method includes reacting a salt of a cation and an organic acid in a solution with nitric acid whereby the organic acid is acidified and a salt of the cation and nitrate is formed. The cation is capable of forming an insoluble carbonate salt. In a particular embodiment, the step of reacting the cation/organic acid salt with nitric acid can include contacting the cation/organic acid salt with an ion exchange resin to acidify the organic acid. This embodiment further includes regenerating the ion exchange resin with nitric acid to form a salt of the cation captured by the ion exchange resin with nitrate. The method further includes recovering the organic acid from the solution. Finally, the cation/nitrate salt is reacted with ammonium carbonate to form ammonium nitrate and an insoluble carbonate salt.

In particular embodiments, the organic acid can be selected from acetic acid, lactic acid, succinic acid, propionic acid, butyric acid, citric acid, benzoic acid, sorbic acid, tartaric acid, malic acid, gluconic acid, and fumaric acid. In addition, the organic acid salt can be produced by fermentation in a fermentation medium. In this embodiment, the fermentation medium can be neutralized by addition of an insoluble carbonate salt, which can be calcium carbonate. This step of neutralizing can either include addition of calcium carbonate or the addition of calcium oxide that is produced by calcination of calcium carbonate.

The cation in the process can be selected from calcium, magnesium, barium, strontium and zinc. Further, the step of recovering the organic acid from the solution can include distillation, extraction, reactive separation, crystallization, stripping and dialysis. The present method can further include the step of processing the ammonium nitrate produced by the process into a fertilizer product.

In another embodiment, a method for production of an organic acid and ammonium nitrate is provided. This method includes conducting a fermentation to produce an organic acid salt in a fermentation medium. The medium is neutralized with a carbonate that comprises a cation that can form an insoluble carbonate salt, whereby a salt comprising the cation and the organic acid is formed. The method further includes acidifying the cation/organic acid salt with nitric acid to form an acidified organic acid in a salt of the cation and nitrate. The organic acid is recovered from the solution. The method finally includes reacting the cation/nitrate salt with ammonium carbonate to form ammonium nitrate and an insoluble carbonate salt.

In a further embodiment of the present invention, a method for production of an organic acid and ammonium nitrate is provided. This method includes conducting a fermentation to produce a salt of an organic acid wherein the organic acid is selected from acetic acid, lactic acid, succinic acid, propionic acid, butyric acid, citric acid, benzoic acid, sorbic acid, tartaric acid, malic acid, gluconic acid, and fumaric acid. The method further includes neutralizing the fermentation medium with calcium carbonate whereby a calcium/organic acid salt is formed. The calcium/organic acid salt is acidified with nitric acid to form the acidified organic acid and calcium nitrate. The organic acid is recovered from the medium and the calcium nitrate is reacted with ammonium carbonate to form ammonium nitrate and calcium carbonate. Finally, the ammonium nitrate is processed into a fertilizer product.

### BRIEF DESCRIPTION OF THE DRAWINGS

FIG. 1 is a block flow diagram of a conventional nitrogen fertilizer process.

FIG. 2 is a comparison of various cations used in fermentation.

FIG. 3 is a block flow diagram of the production of ammonium nitrate from calcium nitrate and ammonium carbonate.

FIG. 4 is a block flow diagram of one embodiment of the present invention.

### DETAILED DESCRIPTION OF THE INVENTION

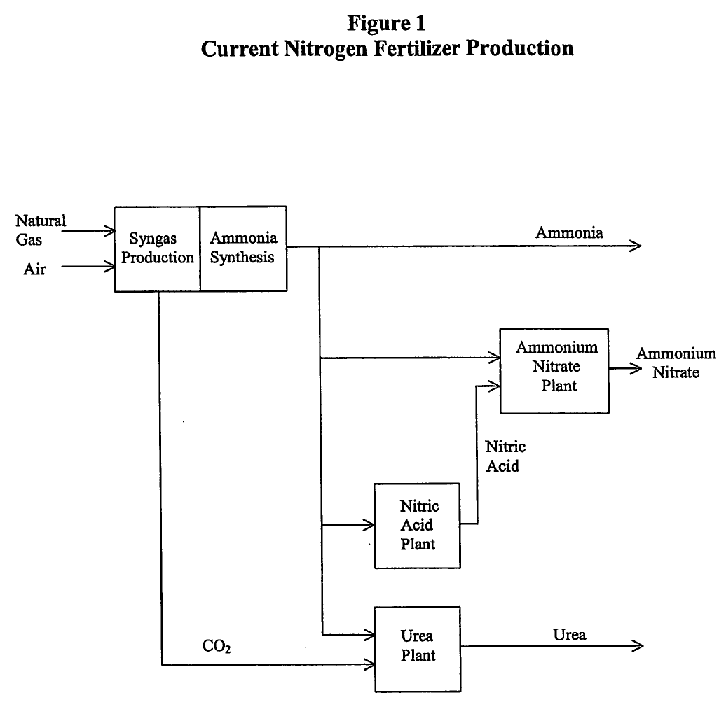

The present invention provides a means for the integration of the recovery of organic acids from dilute salt solution, such as those produced by fermentation, with the production of nitrogen fertilizers in order to capture wasted chemical energy from the fertilizer process and at the same time produce a valuable fertilizer ingredient. Production of un-utilized byproduct salts can be minimized. In the production of ammonium nitrate, chemical energy is wasted by the reaction of nitric acid and ammonia as shown in FIG. 1. In the present invention, this chemical energy is captured to provide the chemical energy required to acidify organic acid salts to provide for their recovery, for example, from fermentation broths. The basic process of this invention can use a dilute salt solution of the organic acid and a suitable cation produced by fermentation of any suitable substrate.

The process includes the steps of:
A. Reaction of an organic acid salt with nitric acid to acidify the organic acid and produce a soluble nitrate salt solution of the cation from the organic acid salt,
B. Removal of the organic acid from the solution by any suitable means, and
C. Reaction of the nitrate salt with ammonium carbonate to produce an insoluble carbonate salt and ammonium nitrate.

The carbonate salt is preferably returned to a fermentation either as the carbonate, or after calcination to produce an oxide, to provide the base required to neutralize an organic acid produced in the fermentation, to keep a near neutral pH, and to provide the organic acid salt. The ammonium nitrate can be processed in the conventional manner to nitrogen fertilizer.

Organic Acid Salt Solution

While the preferred organic acid salt solution of the present invention is produced by fermentation, such dilute solutions can be obtained from other sources, such as the production of byproduct acetate salts in the production of cellulose acetate and others.

The production of organic acids by fermentation is widely known for the production of many important organic acids such as acetic acid, lactic acid, succinic acid, propionic acid, butyric acid, citric acid, benzoic acid, sorbic acid, tartaric acid, malic acid, gluconic acid, and fumaric acid. Citric acid and lactic acid are produced by fermentation at industrial scale. Production of organic acids can be for direct use in industrial processes, or as food ingredients, for example. The production of organic acids as intermediates for other products has been proposed, such as the production of poly(lactic acid), or PLA, from lactic acid. The use of acetic acid as an intermediate for the production of ethanol has been proposed in U.S. Pat. No. 6,509,180, which is incorporated herein its entirety.

During a typical fermentation for the production of organic acids, it is common to introduce a base for neutralization of the fermentation medium so that the fermentation can be conducted at near-neutral pH. Since the pKa of many important organic acids are well below neutral, the organic acid will occur in the form of a salt formed with the cation of the neutralizing base. Such a cation/organic acid salt is then typically acidified so that the organic acid occurs in its protonated acid form for ease of recovery, as described below in more detail.

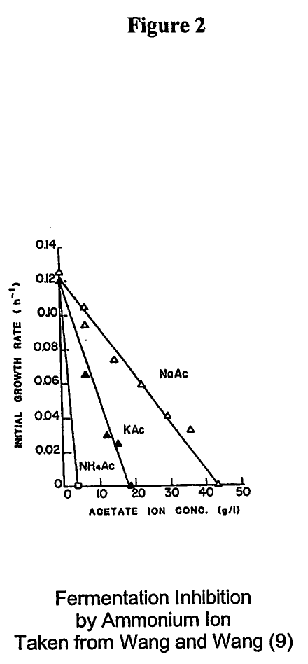

It would be possible to directly co-produce ammonium nitrate by using ammonia as the neutralizing base in the fermentation step for some fermentations, and then nitric acid for acidification. However, while low levels of ammonia can be used as a nitrogen source for these fermentations, the high levels that would be generated if ammonia were the neutralizing agent are inhibitory in others. For example, FIG. 2 shows the growth rate of Clostridium thermoaceticum DSM 521 at various levels of sodium acetate, potassium acetate or ammonium acetate present in the media. The ammonium ion is clearly inhibitory. (Ref. Wang, G., Wang, D. I., “Elucidation of Growth Inhibition and Acetic Acid Production by Clostridium thermoaceticum”, Applied and Environmental Microbiology, Vol. 17, No. 2, p. 294-298, 1984).

However, other cations, such as calcium, are typically not inhibitory. For example, if a fermentation producing acetic acid is neutralized with calcium carbonate, the resulting organic acid salt produced in fermentation will be calcium acetate:
2 Acetic Acid+CaCO₃→Calcium Acetate+H₂O+CO₂

The preferred cations for organic acid salts of the present invention are those that provide soluble organic acid salts up to at least the limit of the concentration of the acid that can be produced in a fermentation, that are not toxic to the organisms in a fermentation, and that are not inhibitory to a fermentation. In addition, the cation forms an insoluble carbonate salt when the cation/nitrate salt is reacted with ammonium carbonate to form ammonium nitrate and a carbonate salt. In this manner, the carbonate salt precipitates from solution and is thus easily recovered for recycle back to the fermentation step, as discussed below in more detail. For example, the cation/carbonate salt, at 25° C., typically has a water solubility of less than about 1 gram/100 cc water, less than about 0.1 gram/100 cc water, and less than about 0.01 gram/100 cc water. Alternatively, upon formation of the carbonate salt, at least about 80% of the cation is precipitated in the form of the carbonate salt, more preferably at least about 90%, and more preferably at least about 95%. For example, calcium lactate is soluble up to about 7.9% at 30° C. Calcium nitrate is very water soluble, 129.3 g in 100 g of water at 20° C. Calcium carbonate has very low water solubility, 0.00153 grams/100 cc of water at 25° C. Suitable examples of cations are calcium, magnesium (carbonate has a solubility of 0.0106 grams/100 cc), barium (carbonate has a solubility of 0.002 grams/100 cc), strontium (carbonate has a solubility of 0.0011 grams/100 cc) and zinc (carbonate has a solubility of 0.001 grams/100 cc).

The concentration of the cation/organic acid salt in solution depends on the source of the solution. For example, if the organic acid is the product of a microbial fermentation, the concentration of the acid, and the corresponding organic acid salt, is simply the concentration of the acid that is produced in the fermentation.

Acidification of the Organic Acid

The present invention further includes reacting the cation/organic acid salt in a solution with nitric acid to acidify the organic acid and form a salt of the cation and nitrate. The amount of nitric acid added to the salt solution is dependent upon the concentration of the organic acid salt. Sufficient nitric acid is added to acidify the organic acid salt. For example, in the case of calcium acetate, acidification of the calcium acetate directly with nitric acid produces calcium nitrate, Ca(NO₃)₂:
Calcium Acetate+2 HNO₃→2 Acetic Acid+Ca(NO₃)₂

Other organic acids react in a completely analogous manner. Calcium nitrate is very water soluble, 129.3 g in 100 g of water at 20° C.

In a specific embodiment, acidification of the organic acid is accomplished by contacting the cation/organic acid salt in a solution with an ion exchange resin in its acid form. The organic acid is acidified and the cation is captured by the resin. The resin is then regenerated with nitric acid to form a salt of the cation and nitrate. (Ref. Bailey, et al, “Production of Lactic Acid by Continuous Fermentation using an Inexpensive Raw Material and a Simplified Method of Lactic Acid Purification”, Oct. 6, 1987, U.S. Pat. No. 4,698,303).

Recovery of Organic Acids

Once the organic acid has been produced in solution in its protonated form by direct acidification, any suitable means can be used for its recovery from the broth (Donald F. Othmer, “Acetic Acid Recovery Methods”, Chemical Engineering Progress, Vol. 54, No. 7, July, 1958; Busche, R. M., “Recovering Chemical Products from Dilute Fermentation Broths”, Biotechnology and Bioengineering Symp. No. 13, p. 597-615, 1983). For example, such methods include distillation, extraction, reactive separation, crystallization, stripping and dialysis.

Solvent extraction of organic acids from dilute solution has been studied in detail. (Ref. James M. Wardell and C. Judson King, “Solvent Equilibrium for Extraction of Carboxylic Acids from Water”, Journal of Chemical and Engineering Data, Vol. 23, No. 2, 1978; N. L. Ricker, J. N. Michaels, and C. Judson King, “Solvent Properties of Organic Bases for Extraction of Acetic Acid from Water”, Journal of Separation Process Technology, Vol. 1, No. 1, 1979; N. L. Ricker, E. F. Pittman, and C. Judson King, “Solvent Extraction with Amines for Recovery of Acetic Acid from Dilute Aqueous Industrial Streams”, Journal of Separation Process Technology, Vol. 1, No. 2, 1980; Baniel, A. M., Eyal, A. M., Mizrahi, J., Hazan, B., Fisher, R. R., Kolstad, J. J., Steward, B. F., “Lactic Acid Production, Separation, and/or Recovery Process”, U.S. Pat. No. 5,510,526, Apr. 23, 1996).

Reactive separation has been proposed as a useful means of separation of certain organic acids from dilute solution (Ref. Scates, M. O., Parker, S. E., Lacy, J. B., Gibbs, R. K., “Recovery of Acetic Acid from Dilute Aqueous Streams Formed During a Carbonylation Process”, U.S. Pat. No. 5,599,976, Feb. 4, 1997; Xu, Z. P., Chuang, K. T., “Kinetics of Acetic Acid Esterification over Ion Exchange Catalysts”, Canadian Journal of Chemical Engineering, Vol. 74, p. 493-500, August, 1996; Popken, T., Gotze, L., Gmehling, J., “Reaction Kinetics and Chemical Equilibrium of Homogeneously and Heterogeneously Catalyzed Acetic Acid Esterification with Methanol and Methyl Acetate Hydrolysis”, Industrial and Engineering Chemistry Research, Vol. 39, No. 7, p. 2601-2611, 2000; Gorak, A., Hoffmann, A., “Catalytic Distillation in Structured Packings: Methyl Acetate Synthesis”, AICHE Journal, Vol. 47, No. 5, p. 1067-1076, May, 2001). Reactive distillation is a viable means for recovery of certain organic acids from dilute aqueous solutions. For example, acetic acid has a normal boiling point of 117.9° C., which means that acetic acid is less volatile than water in a binary water+acetic acid mixture. However, ethyl acetate, has a normal boiling point of 77.1° C. Ethyl acetate is produced by the esterification of ethanol and acetic acid:
Acetic Acid+Ethanol⇄Ethyl Acetate+H₂O

This reaction is an equilibrium reaction that can be driven to nearly 100% of theoretical yield if either the ethyl acetate or the water products are separated from the reaction mixture as the reaction proceeds. This means that is it possible to feed a reactive distillation column with a dilute acetic acid solution and ethanol and produce ethyl acetate in the distillate and water as the bottoms product. Thus reactive distillation removes the desired product overhead, which saves on energy costs since all of the water in the feed does not have to be vaporized.

Conversion of Nitrate Salt to Ammonium Nitrate

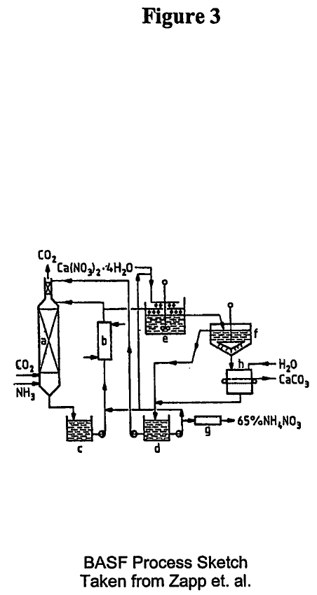

The removal of the organic acid will leave a cation/nitrate salt solution in water. The present invention further includes reacting the cation/nitrate salt with ammonium carbonate to form ammonium nitrate and an insoluble carbonate salt. As noted above, the insoluble carbonate salt can be readily recovered from the solution as a precipitate. Thus, the solid precipitate can be easily separated from the solution by any convenient solid/liquid separation process. The cation/nitrate salt can be readily converted to ammonium nitrate by adapting processes originally developed as variations on the Odda process used in the production of nitrophosphate fertilizers. For example, FIG. 3 is a sketch of one such variation, known as the BASF process for converting calcium nitrate into ammonium nitrate. Calcium nitrate, either in solution or as a solid tetrahydrate, is reacted with a carbonated ammonia solution to produce ammonium nitrate and calcium carbonate according to the reaction: (Ref. Zapp, K. H., Wostbrock, K. H., Sato, K., Zwick, W., Mayer, D., “Ammonium Compounds” in Ullmann's Encyclopedia of Industrial Chemistry, 6th ed., Vol. A2, p. 243-265).
Ca(NO₃)₂+2NH₃+CO₂+H₂O→2NH₄NO₃+CaCO₃

Calcium carbonate has very low water solubility, 0.014 g/l at 25° C. and 1 atm for the calcite form. Nearly 100% of the calcium is precipitated and thus it is easily recovered for recycle back to the fermentation step. The ammonium nitrate can then be further processed into a saleable fertilizer product by known processes.

The ammonia and carbon dioxide required for this process are readily available at a nitrogen complex. The BASF process produces the carbonated ammonia solution in a separate step from the ammonium nitrate production step in order to facilitate heat integration. Other flowsheets and reactor configurations are possible. For example, Hoechst has developed a special vertical reactor that combines the absorption tower and reactor (i.e. equipment items a and e in FIG. 3) into a single vessel. (Ref. Langhans, G., Bieniok, B., ISMA 1976 Technical Conference The Hague, Elsevier, p. 215-233, 1976.)

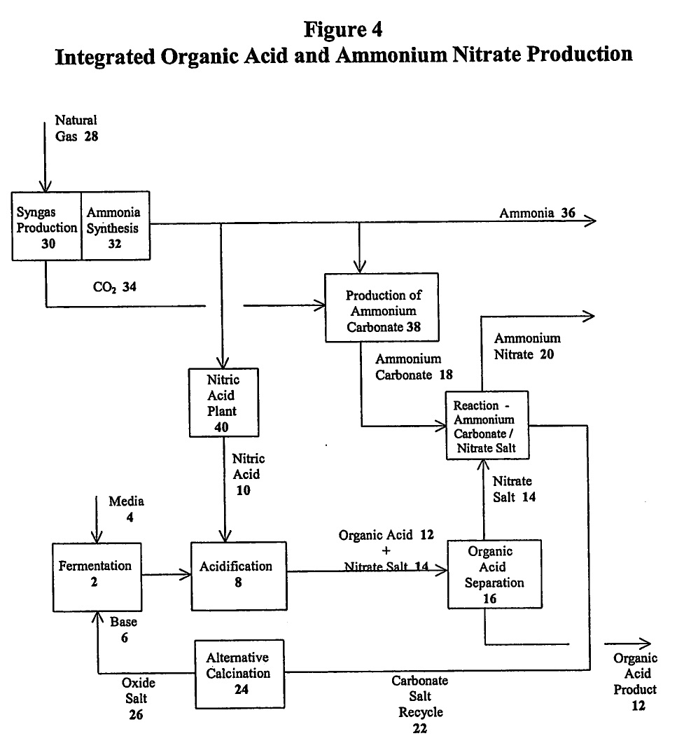

With reference to FIG. 4, one embodiment of the present invention is illustrated. A fermentation 2 is conducted for the production of an organic acid. The fermentation is conducted with the addition of media 4 and base 6 to neutralize the fermentation media to allow for growth of microorganisms at near neutral pH. After the fermentation 2, the media is acidified 8 to form the protonated form of the organic acid. The acidification 8 is conducted by the addition of nitric acid 10. Resulting from the acidification 8 is the organic acid 12 and nitrate salt 14, which is then subjected to an organic acid separation 16. The separation can be conducted by any process known in the art to produce an organic acid product 12. The nitrate salt 14 is then reacted with ammonium carbonate 18. The resulting products are ammonium nitrate 20, which can then be further processed by conventional processes into a fertilizer product and an insoluble carbonate salt 22. Optionally, the carbonate salt can be calcined 24 to produce an oxide salt 26 that is used as the neutralizing base 6 in the fermentation 2.

The nitric acid 10 and ammonium carbonate 18 feeds into this process can come from a nitrogen complex which begins with the processing of natural gas 28 for the production of syngas 30 and synthesis of ammonia 32. Carbon dioxide 34 is produced by the syngas production process 30. The carbon dioxide 34 in conjunction with ammonia 36 produced by the ammonia synthesis 32 are used for the production of ammonium carbonate 38. In addition, ammonia 36 is used as a feed to a nitric acid production plant 40 for the production of nitric acid 1.

This application claims priority from U.S. Provisional Patent Application Ser. No. 60/439,148, filed Jan. 10, 2003, which is incorporated herein by reference in its entirety.

While various embodiments of the present invention have been described in detail, it is apparent that modifications and adaptations of those embodiments will occur to those skilled in the art. However, it is to be expressly understood that such modifications and adaptations are within the scope and spirit of the present invention.

What is claimed is:

1. A method for production of an organic acid and ammonium nitrate, comprising:
a. reacting a cation/organic acid salt in a solution with nitric acid to acidify the organic acid and form a salt of the cation and nitrate, wherein the cation can form an insoluble carbonate salt;
b. recovering the organic acid from the solution; and
c. reacting the cation/nitrate salt with ammonium carbonate to form ammonium nitrate and an insoluble carbonate salt.

2. The method, as claimed in claim 1, wherein the organic acid is selected from the group consisting of acetic acid, lactic acid, succinic acid, propionic acid, butyric acid, citric acid, benzoic acid, sorbic acid, tartaric acid, malic acid, gluconic acid, and fumaric acid.

3. The method, as claimed in claim 1, wherein the organic acid salt is produced by fermentation in a fermentation medium.

4. The method, as claimed in claim 3, wherein the fermentation medium was neutralized with the insoluble carbonate salt.

5. The method, as claimed in claim 4, wherein the cation is calcium and the insoluble carbonate salt is calcium carbonate.

6. The method, as claimed in claim 5, wherein the step of neutralizing is selected from the group consisting of addition of calcium carbonate to the fermentation medium and addition of calcium oxide produced by calcination of calcium carbonate to the fermentation medium.

7. The method, as claimed in claim 1, wherein the cation is selected from the group consisting of calcium, magnesium, barium, strontium and zinc.

8. The method, as claimed in claim 1, wherein the step of recovering is selected from the group consisting of distillation, extraction, reactive separation, crystallization, stripping and dialysis.

9. The method, as claimed in claim 1, further comprising processing the ammonium nitrate into a fertilizer product.

10. The method, as claimed in claim 1, wherein the step of reacting a cation/organic acid salt in a solution with nitric acid comprises contacting the cation/organic acid salt in a solution with an ion exchange resin to acidify the organic acid and regenerating the ion exchange resin with nitric acid to form a salt of the cation and nitrate.

11. A method for production of an organic acid and ammonium nitrate, comprising:
a. conducting a fermentation to produce an organic acid salt in a fermentation medium;
b. neutralizing the fermentation medium with a carbonate comprising a cation that can form an insoluble carbonate salt, whereby a salt comprising the cation and the organic acid is formed;
c. acidifying the cation/organic acid salt with nitric acid to form an acidified organic acid and a salt of the cation and nitrate;
d. recovering the organic acid; and
e. reacting the cation/nitrate salt with ammonium carbonate to form ammonium nitrate and an insoluble carbonate salt.

12. The method, as claimed in claim 11, wherein the organic acid is selected from the group consisting of acetic acid, lactic acid, succinic acid, propionic acid, butyric acid, citric acid, benzoic acid, sorbic acid, tartaric acid, malic acid, gluconic acid, and fumaric acid.

13. The method, as claimed in claim 11, wherein the insoluble carbonate salt is selected from the group consisting of calcium carbonate, magnesium carbonate, barium carbonate, strontium carbonate and zinc carbonate.

14. The method, as claimed in claim 11, wherein the insoluble carbonate salt is calcium carbonate.

15. The method, as claimed in claim 14, wherein the step of neutralizing is selected from the group consisting of addition calcium carbonate to the fermentation medium and addition of calcium oxide produced by calcination of calcium carbonate to the fermentation medium.

16. The method, as claimed in claim 11, wherein the step of recovering is selected from the group consisting of distillation, extraction, reactive separation, crystallization, stripping and dialysis.

17. The method, as claimed in claim 11, wherein the insoluble carbonate salt formed by reacting the cation/nitrate salt with ammonium carbonate is used in the step of neutralizing.

18. The method, as claimed in claim 11, further comprising processing the ammonium nitrate into a fertilizer product.

19. The method, as claimed in claim 11, wherein the step of acidifying the cation/organic acid salt with nitric acid comprises contacting the cation/organic acid salt with an ion exchange resin to acidify the organic acid and regenerating the ion exchange resin with nitric acid to form a salt of the cation and nitrate.

20. A method for production of an organic acid and ammonium nitrate, comprising:
a. conducting a fermentation to produce a salt of an organic acid selected from the group consisting of acetic acid, lactic acid, succinic acid, propionic acid, butyric acid, citric acid, benzoic acid, sorbic acid, tartaric acid, malic acid, gluconic acid, and fumaric acid in a fermentation medium;
b. neutralizing the fermentation medium with calcium carbonate, whereby a calcium/organic acid salt is formed;
c. acidifying the calcium/organic acid salt with nitric acid to form an acidified organic acid and calcium nitrate;
d. recovering the organic acid;
e. reacting the calcium nitrate with ammonium carbonate to form ammonium nitrate and calcium carbonate; and
f. processing the ammonium nitrate into a fertilizer product.
